(19) United States
(12) Patent Application Publication
(10) Pub. No.: US 2021/0259807 A1
(43) Pub. Date: Aug. 26, 2021

(54) METHOD FOR EVALUATING A DENTAL SITUATION

(71) Applicant: DENTAL MONITORING, Paris (FR)

(72) Inventors: Philippe SALAH, Bagnolet (FR); Thomas PELLISSARD, Clichy (FR); Guillaume GHYSELINCK, Cantin (FR); Laurent DEBRAUX, Paris (FR)

(73) Assignee: Dental Monitoring, Paris (FR)

(21) Appl. No.: 17/053,819

(22) PCT Filed: May 7, 2019
(86) PCT No.: PCT/EP2019/061651

§ 371 Date: Nov. 9, 2020

(30) Foreign Application Priority Data
May 9, 2018 (EP) 18305571.4
May 25, 2018 (EP) 18305637.3

(51) Int. Cl. A61C 7/00; A61C 9/00; G06T 7/00; G06N 3/08

(52) U.S. Cl. CPC A61C 7/002; A61C 9/0053; G06T 2207/20084; G06N 3/08; G06T 2207/30036; G06T 7/0012

(57) ABSTRACT

Method for preparing a composed response from a value for at least one dental attribute relating to the dental situation of a patient. The method includes: (d) selection by a composition computer of at least one relevant standard response from a base or standard responses and as a function of the value of the at least one dental attribute; (e) composition of a composed response by the composition computer on the basis of the at least one relevant standard response; (f) optionally, validation and/or modification of the composed response by a controller; (g) presentation of the optionally validated and/or modified composed response. The selection in (d) and the composition in (e) are determined according to composition rules of a composition protocol. Before (e), the composition protocol is selected in a protocol models base and/or is created by selecting a protocol model in a protocol models base and then modifying.

### TECHNICAL FIELD

The present invention concerns a method for evaluating a dental situation.

### PRIOR ART

It is desirable for everyone to have a regular dental checkup, in particular in order to evaluate their dental situation, that is to say in particular to verify that the position or the appearance of the teeth is not evolving in an unfavorable manner or that an orthodontic device is in good condition, or correctly positioned. During an orthodontic treatment that unfavorable evolution can in particular lead to modifying the treatment. After an orthodontic treatment, this so-called “recidivist” unfavorable evolution may lead to further treatment. Finally, more generally and independently of any treatment, everyone may wish to evaluate if their dental situation, and in particular their orthodontic situation, is satisfactory.

Checkups are conventionally carried out by a dental care professional, in particular an orthodontist or a dentist, alone having appropriate equipment. These checkups are therefore costly. Also, visits to the dental care professional are stressful.

FR 3 010 629 describes a method of checking the positions of the teeth of a patient. Practical information can be generated, in particular to advise the necessity for urgent consultation of a dentist or an orthodontist.

To limit the number of visits, the Applicant has developed a method enabling, on the basis of
a three-dimensional model of a dental arch of a patient produced before the treatment, termed a “initial reference model”, and then
a simple “updated” image of the arch captured during the treatment, for example from a photograph taken by the patient using their mobile telephone, accurate evaluation of the positions of the teeth at the moment of acquiring the updated image.

That method consists in an iterative process whereby, on each iteration, teeth models of the initial reference model are moved, after which optimum observation conditions of the initial model thus modified (termed a “reference model to be tested”) are determined, the optimum observation conditions being defined as the conditions enabling observation of the reference model to be tested in such a manner that the view of said model is as close as possible to the updated image. A succession of “reference models to be tested” is tested until a maximum level of correspondence is obtained between a reference model to be tested and the updated image. This last reference model to be tested is then considered as representing the teeth in their position at the moment of acquisition of the updated image. This so-called “updated reference model” is ideally a digital three-dimensional reference model from which the updated image could have been captured if that model had been real. In practise, it effectively represents, with great accuracy, the teeth in their position at the moment of acquiring the updated image.

Comparing the initial and updated reference models then enables evaluation of the dental situation of the patient. The dental situation is conventionally evaluated by a centralized computer processing remotely requests sent by the patients using their mobile telephones and containing the updated images. This process is described in particular in PCT/EP2015/074896.

The Applicant has also developed a method employing an artificial intelligence algorithm, preferably a neural network, and enabling, on the basis of a simple updated image of the arch of the patient, for example a photograph taken by the patient using their mobile telephone, evaluation of the dental situation of the patient. To this end, the neural network is trained beforehand by means of a learning base typically including more than 10 000, preferably more than 50 000, more preferably more than 100 000 records, each record containing a historical image, preferably a photo, of a “historical” dental arch and a “historical” description supplying values for the attributes of the historical image, in particular for attributes enabling evaluation of the dental situation of the patient.

Like the method described in PCT/EP2015/074896, the evaluation of the dental situation by artificial intelligence is preferably carried out on a centralized computer processing remotely requests sent by the patients using their mobile telephones and containing the updated images.

The two methods of the Applicant described hereinabove advantageously limit the number of visits to the dental care professional and do not necessitate costly dedicated equipment.

However, the patients to whom these methods are offered are not using them sufficiently. In particular, it is found that numerous patients cease to send requests after a few weeks,

There is therefore a need for a method improving the level of participation of the patients.

Moreover, there is a permanent need to train the patients in good hygiene practises, in particular brushing the teeth.

Finally, evaluation of the dental situation by the centralized computer is not always well matched to the requirements of the dental care professional. In particular, it may be of no utility if the dental situation is satisfactory. It may also be insufficiently detailed for them to be able to establish a diagnosis or a recommendation.

An objective of the present invention is to respond at least in part to those requirements.

### SUMMARY OF THE INVENTION

The invention provides a method for evaluating a dental situation of a target patient, preferably monitored by a dental care professional, said method including the following steps:
a) acquisition by means of a mobile telephone of at least one image representing a dental arch of the target patient, termed an “updated image”;
b) preparation by the mobile telephone of a request including the updated image and transmission of the request to an analysis computer;
c) analysis of the request by the analysis computer in such a manner as to determine a value for at least one dental attribute relating to the dental situation of the target patient and transmission of the value of the dental attribute to a composition computer;
d) selection by the composition computer of at least one relevant standard response from a base of standard responses and as a function of said value of said at least one dental attribute;
e) composition of a composed response by the composition computer on the basis of said at least one relevant standard response;
f) optionally, validation and/or modification of the composed response by a controller;
g) presentation of the composed response, optionally validated and/or modified; the selection in step d) and the composition in step e) being determined according to composition rules of a composition protocol.

In accordance with a first principal aspect of the invention, in step g) the composition computer transmits the composed response, optionally validated and/or modified, to the mobile telephone and presents the composed response on the telephone, optionally validated and/or modified.

As will emerge in more detail in the remainder of the description, a method in accordance with the first principal aspect of the invention enables a personalized but automatic response to the target patient.

In particular, the inventors have discovered that patients cease to send requests when they receive no response and that dental care professionals do not always respond to all the requests that they receive. By systematizing the response procedure, the level of participation of the patients increases significantly.

The inventors have also observed that the insertion of advice into the composed responses advantageously makes it possible to educate patients in good practises, and in particular to improve oral and dental hygiene.

The composition protocol may be common to all the patients of a group of patients of the dental care professional or be specific to the patient.

In accordance with a second principal aspect of the invention, in step g) the composition computer transmits and presents to the dental care professional the composed response, optionally validated and/or modified.

The composed response may advantageously consist in a medical summary and preferably include advice for the dental care professional matched to the medical summary

As will emerge in more detail in the remainder of the description, a method in accordance with the second principal aspect of the invention enables a personalized but automatic response to the dental care professional. In defining the composition protocol the dental care professional can for example:
decide on thresholds from which they require a composed response to be sent to them, and/or
define the level of detail that they require in the composed response, and/or
decide if the composed response should include medical recommendations, and/or
decide on information that they wish to highlight in the composed response, and/or
define one or more other addressees of the composed response, for example to accelerate ordering an orthodontic item or to prepare for the next appointment with the target patient.

In accordance with a third principal aspect of the invention, in step g) the composition computer transmits and presents to a third party, that is to say a person different from the patient and the dental care professional who is monitoring the target patient, the composed response, optionally validated and/or modified.

The third party may in particular be an insurance professional. The target patient may be a person insured by the insurance professional or requesting to be insured by the insurance professional.

The composed response may advantageously consist in a medical summary

As will emerge in more detail in the remainder of the description a method in accordance with the third principal aspect of the invention enables the insurance professional to receive very easily and rapidly a summary of the situation of the patient.

They are therefore able to verify that information supplied to them by the target patient independently of the use of the method, for example by means of a questionnaire, conforms to the composed response. In one embodiment the information contained in the composed response is used to establish an insurance policy proposal.

The composition computer preferably also composes, as a function of the composed response addressed to the insurance professional, a composed response intended for the target patient and/or a composed response intended for a dental care professional who is monitoring the target patient and/or a composed response intended for a dental care professional who is not necessarily monitoring the target patient, but who is determined as a function of the appropriateness of their skills to a dental treatment requirement determined on the basis of the composed response addressed to the insurance professional.

These composed responses are addressed to the target patient, to the dental care professional and to the dental care professional determined as a function of the composed response addressed to the insurance professional, respectively.

In one embodiment the insurance professional
prepares, as a function of the composed response, an insurance policy proposal and/or a request for additional examination and/or a recommendation of a dental care professional and/or a proposed next appointment with the target patient and/or advertising, and
addresses it to the target patient and/or to a dental care professional.

A method in accordance with the invention, in particular according to the principal aspects, may further have one or more of the following optional features:
the analysis computer is programmed, in step c):
   to measure the value of the dental attribute in the updated image; and/or
   to segment the updated image and measure the value of the dental attribute over a segment of the updated image; and/or
   on the basis of the updated image, to look for additional information, preferably additional information specific to the patient, and to determine the value of the dental attribute on the basis of said additionally information;
the analysis computer is programmed, in step c), to determine a three-dimensional digital module of the dental arch, termed an “updated reference model”, observable in a view that can be superimposed on and in register with the updated image, and then to determine the value of the dental attribute from measurements on the updated reference model;
preferably
   by means of an optimization method, preferably a metaheuristic, preferably evolutionist, method, preferably by simulated annealing, and/or
   by means of a neural network, the updated reference model is preferably determined by deformation of an initial reference model, preferably generated by a scan of the dental arch;
the value of the dental attribute qualifies or quantifies the general dental situation of the target patient and/or an orthodontic situation;
the value of the dental attribute qualifies or quantifies a state of an orthodontic device represented in the updated image and/or a state of a tooth of the target patient represented in the updated image and/or the oral-dental hygiene of the patient;
before step e), the dental care professional selects the composition protocol in a base of parameter protocols and/or creates the composition protocol by selecting a composition protocol in a base of parameter protocols and then modifying it;
before step e), the dental care professional configures the composition protocol in such a manner as to select a region of the mouth of the patient so that the analysis in step c) and/or the selection of the relevant standard response in step d) apply/applies only to said region;
in step d) the computer selects at least one standard response as a function of data relating to the target patient that cannot be determined by the analysis of the dental situation in step c);
before step d) the composition computer evaluates
   the reaction of the target patient, in particular in accordance with the first principal aspect of the invention, and/or
   the reaction of the dental care professional, in particular in accordance with the second principal aspect of the invention,
      after of an earlier composed response generated in an earlier step g) is sent, and then, in step d), selects said standard response as a function of the evaluation of said reaction;
the earlier composed response is a response generated during an earlier cycle of steps a) to g) on the basis of an earlier updated image or is a response generated in an earlier step g) relating to said updated image;
at least one composition rule is configured to operate on said composition as a function of said at least one relevant standard response selected in step d), and/or as a function of data relating to the target patient and/or as a function of data relating to the dental care professional;
the composition rule is configured to operate on said composition as a function of data relating to the target patient that cannot be determined by the analysis of the dental situation in step c);
at least one composition rule is chosen in the group formed by:
   a filter determining if a standard response can or cannot be integrated into the composed response;
   a composition rule imposing a format on the relevant standard response, preferably imposing a page background and/or a font and/or a font size and/or a font color and/or a translation into a language or a language register;
   a composition rule determining a position of each relevant standard response as a function of its content or of a category to which it belongs;
   a conditional composition rule;
the conditional composition rule:
   has an effect that depends on the dental situation of the target patient, and/or
   has an effect that depends on the standard response to which it applies, and/or
   modifies the relevant standard response or the composed response as a whole, and/or
   takes account of past events; and/or
   applies to the composed response and depends on data relating to the patient, and/or
   has an effect that depends on earlier exchanges with the target patient, and/or
   has an effect that depends on an arbitrary choice, in particular by the dental care professional monitoring the target patient or an insurance professional;
before step e),
   a dental care professional in accordance with the target patient, or
   an insurance professional, in particular an insurance company, or
   an organization having a centralized computer programmed to execute at least steps d) and e) on the basis of a plurality of requests each containing a value of said at least one dental attribute, preferably sent by a plurality of patients monitored by a plurality of dental care professionals, preferably by means of their mobile telephones,
      selects the composition protocol in said protocol models base and/or creates the composition protocol by selecting a protocol model in a protocol models base and then modifying it;
in step f) the controller is a dental care professional the same as or different from the dental care professional who is monitoring the target patient;
in one embodiment the analysis computer and/or the composition computer is/are integrated into the mobile telephone;
in one embodiment the analysis computer and/or the composition computer is/are remote from the mobile telephone and in particular may be in an evaluation center with which the mobile telephone communicates, the evaluation center preferably computer processing requests coming from patients of more than 1, more than 5, more than 50, more than one 100, preferably more than 1000 dental care professionals;
in accordance with the first principal aspect of the invention the composition computer preferably composes, in accordance with a composition protocol identical to or different from that employed in step d) and in step e), a medical summary as a function of said value of said at least one dental attribute and presents said medical summary to the dental care professional.

Of course, the various principal aspects of the invention are not incompatible. In particular the method may be used, for the same dental situation, to generate a composed response intended for the target patient and another composed response intended for the dental care professional, preferably in accordance with two different composition protocols. For clarity, in the present description “professional” denotes the composition protocol used to generate a composed response intended for the dental care professional.

The invention further concerns a system including a mobile telephone, an analysis computer and a composition computer configured to execute steps a) and b) and optionally g), step c), and steps d) and e), respectively.

The invention also concerns:
a computer program, and in particular a mobile telephone app, including program code instructions for executing
   steps a) and b) and optionally g), and/or
   step c), and/or
   steps d) and e),
      when said program is executed by a computer,
a computing medium on which a program of this type is stored, for example a memory or a CD-ROM, and
   a computer, in particular a personal device, in particular a mobile telephone or a tablet or a computer, into which a program of this kind is loaded.

The invention further provides a method for preparing a response on the basis of a value for at least one dental attribute relating to the dental situation of a target patient, in particular of a target patient monitored by a dental care professional, said method including the following steps:
d′) selection by a composition computer in a standard responses base and as a function of said value of said at least one dental attribute of at least one relevant standard response, preferably a plurality of relevant standard responses;
e′) composition by the composition computer of a composed response on the basis of said at least one relevant standard response;
f′) optionally, validation and/or modification of the composed response by a controller;
g′) presentation of the composed response, optionally validated and/or modified, preferably on a mobile telephone;
   the selection in step d′) and the composition in step e′) being determined in accordance with composition rules of a composition protocol.

Steps d′) to g′) may have one or more features, optional or not, of steps d) to g), respectively. Steps d′) to g′) may be identical to steps d) to g), respectively.

The invention further concerns a system including a composition computer configured to execute steps d′) and e′). The system may also include a mobile telephone configured to execute step g′).

The invention also concerns:
a computer program, and in particular a mobile telephone app, including program code instructions for executing
   step g′), and/or
   steps d′) and e′),
      when said program is executed by a computer,
a computer medium on which is stored a program of this kind, for example a memory or a CD-ROM, and
a computer, in particular a personal device, in particular a mobile telephone or a tablet or a computer, in which a program of this kind is loaded.

### DEFINITIONS

The “representation of a dental arch” is a representation of all or part of said arch.

Unless otherwise indicated, by “model” is meant a digital three-dimensional model. By “model of an arch” is meant a representation of all or part of said arch.

By “target patient” is meant any person for whom a method in accordance with the invention is executed in order to evaluate their dental situation, whether that person is ill or not.

By “mobile telephone” is meant any portable device enabling capture of photos or films and communication via a mobile telephone network.

By “computer” is meant a data processing unit. That unit may in particular be integrated into the mobile telephone or be a PC type computer or a server of an “evaluation center”, for example a computer installed at the surgery of the dental care professional, or be a tablet. The mobile telephone and the computer then include communication means for exchange between them, in particular for transmitting the request including the updated image and/or the composed response.

A computer conventionally includes in particular a processor, a memory, a human-machine interface, conventionally including a screen, an internet, WIFI, Bluetooth® or telephone network communication module. Software configured to execute step c) is loaded into the memory of the analysis computer. Software configured to execute steps d) and e) is loaded into the memory of the composition computer. A computer may also be connected to a printer.

By “image” is meant an image in two dimensions, such as a photograph or an image from a video. An image is formed of pixels.

A “neural network” or “artificial neural network” is a set of algorithms well known to the person skilled in the art. A “learning base” is a base of computer records adapted to train a neural network.

To be operational, a neural network is conventionally trained by a so called deep learning process from a learning base.

The learning base conventionally contains information on the two types of object that the neural network has to learn to match.

Training may be based on a learning base consisting of records each including a first object of a first type and a corresponding second object of a second type. The first objects are present at the input of the neural network and the second objects at the output of the neural network, which enables the neural network to learn to provide, based on any object of the first type, a corresponding object of the second type.

For example, the neural network may be supplied with a set of records each containing an image (first object) and the value of a dental attribute for that image (second object) in such a manner that it determines the values of its parameters so that when an updated image is presented at its input its supplies at its output a value for that dental attribute in that updated image.

Training a neural network is matched to the intended aim and does not pose any particular difficulty for the person skilled in the art.

For example, each record from the learning base may include an image of a dental arch and a description identifying in that image the representations of the teeth, or “tooth zones”, and the corresponding tooth numbers. After being trained, the neural network could therefore identify, in an image presented to it, the representations of the teeth and the corresponding tooth numbers.

Unless otherwise indicated “comprise”, “include” and “feature” must be interpreted in a broad manner that is not limiting on the invention.

### BRIEF DESCRIPTION OF THE FIGURES

Other features and advantages of the invention will become more apparent on reading the following detailed description and examining the appended drawings in which:

FIG. 1 illustrates the execution of a method in accordance with the invention in an embodiment in which the analysis of the request and the composition of the composed response are effected remotely from the mobile telephone,

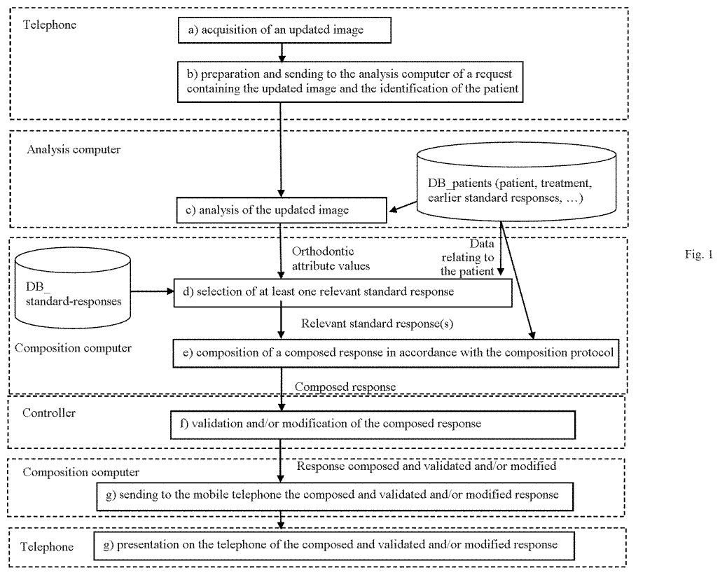

FIG. 2 illustrates the use of a mouth spreader, and

FIG. 3 illustrates the execution of a method in accordance with the invention in an embodiment in which the analysis of the request and the composition of the composed response are effected in the mobile telephone,

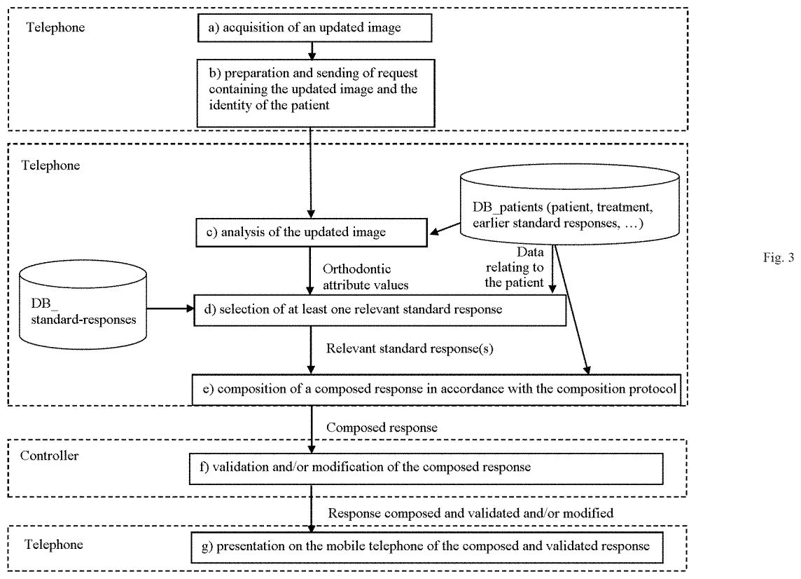

### DETAILED DESCRIPTION OF A PREFERRED EMBODIMENT

A method according to the invention includes the steps mentioned hereinafter. This method is described in detail in the context of the first principal aspect of the invention for presenting a composed response to the target patient.

An objective of the method is then to generate automatically responses to requests from patients. To this end the method uses a composition protocol applicable to the target patient or to a group of patients of the dental care professional that fixes the composition rules governing step e).

The group of patients, or “cohort”, preferably includes more than 20, more than 50, more than one hundred, more than three hundred, more than five hundred, or even more than one thousand patients. In one embodiment the group of patients includes all the patients monitored by the dental care professional. The dental care professional is therefore advantageously able to apply the same procedure whichever patient is concerned.

The cohort is also able to group the patients having the same pathologies, for example all the patients suffering from sleep apnea.

The composition protocol includes a set of composition rules that determine the selection of the relevant standard responses and how the selected relevant standard responses are modified and/or arranged to compose the composed response.

The standard responses base in which the relevant standard responses are selected may include more than 100, more than 500 or more than 1000 standard responses.

In particular it may include messages providing information on the dental situation of the target patient, messages providing a recommendation, for example relating to the brushing of a particular tooth, congratulations or encouragement messages, warning messages, reassuring messages or messages asking the target patient to make an appointment with the dental care professional.

For selecting relevant standard responses a composition rule may be a simple filter determining if an associated standard response can be integrated into a composed response or not.

For the modification of the relevant standard responses a composition rule may for example impose a particular format on a selected standard response, for example impose a page background, a font, a font size or a font color, or impose a translation into a particular language, for example into a known language of the target patient, or to impose a particular language register.

For the arrangement of the relevant standard responses, possibly modified, a composition rule may be simply to display all the relevant standard responses, for example in an email A composition response may equally determine the position of each relevant standard response as a function of its content or of the category to which it belongs.

The composition rule may apply to an associated standard response, to an associated set of standard responses, that is to say to a “category” of standard responses, to all the selected standard responses, or to the composed response.

A composition rule may be conditional or non-conditional.

In particular, its action may depend on data relating to the target patient and in particular their dental situation, in particular their orthodontic situation (or “clinical situation”).

Their action may also depend on one or more other standard responses selected to respond to the reception of an updated image and/or one or more standard responses selected to compose a composed response that was sent previously to the target patient and/or the reaction of the target patient to one or more earlier composed responses sent to him before, preferably in the same orthodontic frame (same period between two orthodontic treatments or the same orthodontic treatment).

In one embodiment said reaction may be evaluated by the analysis of the updated images. In particular a modification in the monitoring of an orthodontic treatment and/or in compliance with recommendations relating to oral hygiene may be visually detectable by analysis of successive updated images.

In one embodiment said reaction may be evaluated by an independent analysis of the updated images, for example by seeing if the target patient has read the response that they received on the last execution of the method.

The action of a composition rule may also depend on the reactions of one or more other “historical” patients to one or more earlier composed responses, generated in accordance with the invention, sent to them before, preferably in the context of orthodontic treatments substantially identical to that of the target patient.

The action of a composition rule may further depend on characteristics of the target patient independent of their dental situation, for example their age or their sex, or the affective closeness between the target patient and the dental care professional, notably to determine whether (in France) to use “tu” or “vous”.

The action of a composition rule may further depend on the history of the target patient, that is to say for example the treatments that they have undergone or requests that they have sent in the past or their behavior during treatment. It may in particular depend on the value, determined during at least one earlier step c), of at least one dental attribute relating to the dental situation of the target patient, that is to say the analysis of updated images from other cycles of steps a) to f).

The action of a composition rule may also depend on data independent of the target patient, or “external data”. For example, it may depend on the period of the year in which the request was received from the target patient, for example to wish a happy new year at the beginning of the month of January.

It may also depend on an arbitrary choice of the dental care professional, in particular their specific choice with regard to communication a propos a particular dental situation or a particular region of the mouth of the patient.

The following examples illustrate the variety of possible composition rules:
a composition rule that depends on the dental situation of the target patient, that is to say on the value of at least one dental attribute, may be for example: “if the dental attribute value is “orthodontic aligner detached from tooth No 13”, select the following relevant standard response: ‘the images that you have sent us have enabled us to detect detachment of your orthodontic aligner on a right canine, on the upper arch”.

A composition rule the effect of which depends on the standard response to which it applies may be for example: “impose red as a color for the only relevant standard responses relating to warnings”.

A composition rule modifying a relevant standard response or the composed response as a whole may be for example: “if the target patient is English, use only standard responses in English”, or “if the target patient is English, translate the composed response into English”.

Other conditional composition rules taking account of past events may be for example: “if the target patient has not responded to the previous reminder, this standard response cannot be selected as a relevant standard response” or “if standard response No 8 has been selected as the relevant standard response to compose the preceding composed response, standard response No 9 may not be selected as the relevant standard response”.

A composition rule that applies to the composed response and that depends on data relating to the patient may be for example: “if the target patient is a man, introduce the composed response by ‘Dear Mr.’ followed by the name of the target patient, in lower case, with a first letter in upper case”.

A composition rule the effect of which depends on earlier exchanges with the target patient may be for example: “if the target patient has not made an appointment despite two successive reminders, select the relevant standard response ‘it is absolutely indispensable that you make an appointment quickly. Your dental situation is deteriorating’”.

A composition rule the effect of which depends on an arbitrary choice by the dental care professional may be for example: “do not integrate into the composed response any alert relating to occlusal interferences” or “do not integrate into the composed response a warning relating to tooth No 12”.

In one embodiment the composition protocol is unique and fixed. However, the dental care professional may preferably choose it in a base of protocol models and/or modify in it the values of the parameters of the composition rules.

In one embodiment at least some of the protocol models are pre-parametered and the dental care professional modifies the values of the parameters, for example as a function of the type of patient and/or of choices that are specific to them and/or specifics of the patient. For example, they may use a protocol model pre-parametered for an adult and modify therein the values of parameters for it to suit a child.

The choices may be specific to the dental care professional. For example, depending on the dental care professionals, the composition protocol must or must not indicate the appearance of malocclusion. Parameter values may therefore be independent of the patient and the treatment and arise from an arbitrary decision by the dental care professional.

To set the values of parameters, a tick box may be associated with each standard response from the standard responses base, activation or deactivation of that box defining if the standard response may or may not be used as a relevant standard response. The value of that tick box is an example of a value of a parameter of a composition rule defining the possibility or the prohibition of using the standard response.

The standard responses are more preferably classified into categories. At least one parameter preferably concerns all the standard responses of a category, that is to say the value of that parameter is applicable to all the standard responses of that category. Parameter setting by the dental care professional is advantageously simplified by this.

When the dental care professional is able to choose the composition protocol in a protocol models base, the protocol models preferably include default values for each of the parameters and the dental care professional can modify the values of parameters.

The dental care professional may advantageously set the parameters of the composition protocol to select a region of the mouth to which the analysis in step c) and/or the composition in step e) have to relate. In particular, the composition protocol is preferably configured to allow a selection of teeth.

The composed response will therefore advantageously not include information on any part of the mouth other than said region. Also, this will accelerate the analysis in step c).

Before step a), a reminder preferably informs the target patient of the necessity to acquire an updated image. This reminder is sent to their mobile telephone, for example in the form of an email, an alert or an SMS.

In step a) an updated image of the dental arch of the target patient is acquired by means of a mobile telephone. Step a) is preferably executed by the target patient or someone close to the target patient. Step a) may advantageously be executed away from the evaluation center, in particular more than 1 km, or more than 5 km from the location of the evaluation center, in particular outside the orthodontic surgery.

The updated image is preferably a photograph, in particular a panoramic photograph. In one embodiment, the updated image is extracted from a film.

The updated image is preferably an extra-oral image.

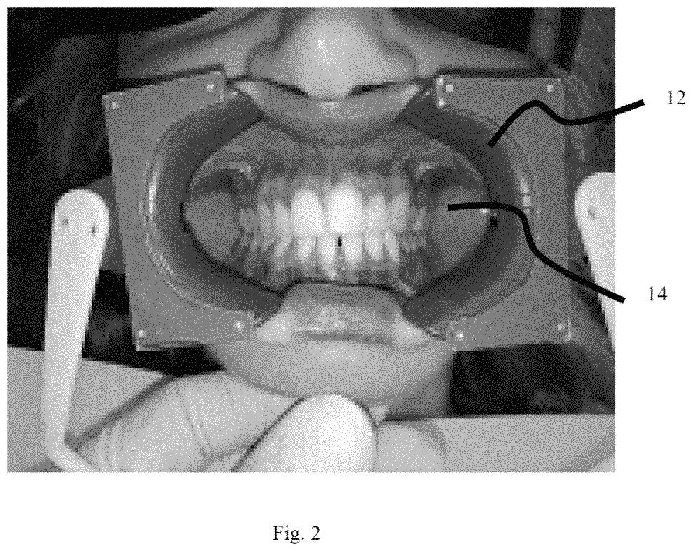

A mouth spreader is preferably used in step a) as represented in FIG. 2. A mouth spreader is a well-known device including channels 12 extending around an opening 14. The target patient places their lips in the channels, which makes it possible to spread them and to improve the observation of the dental arches through the opening. A mouth spreader is preferably supplied to the target patient, for example during an appointment with their orthodontist.

In one embodiment no mouth spreader is used in step a).

In step b) a request is prepared by an app loaded into the mobile telephone. That request includes an updated image acquired in step a) but also information enabling the source of the request to be identified, that is to say an identifier “ID_patient” of the target patient.

The request is processed by an “analysis computer” that may in particular be a processor module integrated into the mobile telephone or a computer of an evaluation center remote from the mobile telephone.

In the latter case the request is transmitted to the analysis computer by the mobile telephone. All known communication means may be used for this purpose. Transmission may in particular be effected via a mobile telephone network to which the mobile telephone subscribes.

The analysis computer is able to receive requests coming from a plurality of target patients of the dental care professional and in particular when they are in an evaluation center, preferably of a plurality of dental care professionals. They preferably receive requests from more than 1000, more than 10 000, preferably more than 50 000, more preferably more than 100 000 target patients.

In step c) the analysis computer analyses the request. In particular it analyses the updated image in order to evaluate the dental situation of the target patient. In one embodiment the evaluation of the dental situation of the target patient is validated and/or modified, in particular added to, by a dental care professional. The dental care professional can also act consequently, for example to order a new orthodontic groove if the evaluation indicates that the one being worn by the target patient is no longer suitable.

The analysis of the updated image may include a measurement of the value of a dental attribute directly on the updated image. The updated image being captured by the patient, the analysis computer can generally not affect any such measurement immediately.

The analysis in step c) therefore preferably includes prior processing of the segmentation of the updated image to determine the elements (or “segments”) that are represented, for example tooth zones, gum zones, or orthodontic device zones.

The value of the attribute may then be measured over the corresponding zone. For example, after identifying the tooth zone of tooth No 3, that is to say the area of the updated image that represents that tooth, the analysis computer is able to measure the color (dental attribute) of that tooth and to determine that it is “normal” (value of the dental attribute).

In a preferred embodiment, on the basis of the updated image, the analysis computer looks for additional information, preferably additional information specific to the patient, and then uses the additional information, preferably in combination with the information as measured on the updated image, optionally after segmentation, to determine a value for the dental attribute.

The analysis computer preferably determines from the updated image a theoretical configuration that corresponds to the updated image and then uses the theoretical configuration to determine the value of the attribute for the updated image.

In particular it may look for a three-dimensional digital model offering a view that can be superposed on and in register with the updated image, that is to say that enables observation of the three-dimensional digital model as the updated image. It is then able to use complementary information from that model, called an “updated reference model”, to determine a value for the dental attribute that is not measurable on the updated image.

For example, if the updated reference mode represents the roots of the teeth, not visible in the updated image, the analysis computer can measure an abnormal movement toward one another of the roots of two adjacent teeth, the dental attribute being “movement toward one another of the roots of adjacent teeth” and its value being “abnormal”.

Analysis of the updated image may in particular employ optimization processes.

To look for the updated reference model it is possible in particular to modify iteratively an initial reference model to obtain an updated reference model that corresponds to the updated image. The updated reference model is therefore ideally a digital three-dimensional reference model from which the updated image could have been captured if that model had been real.

The initial reference model is a three-dimensional digital model of the dental arch of the patient, preferably acquired by means of a scanner, preferably in the surgery of a dental health professional. There is therefore high accuracy.

The search for the updated reference model preferably includes
a first optimization operation making it possible to look for a view of the reference model to be tested that best matches (“best fit”) the updated image; and
a second optimization operation making it possible, by testing a plurality of said reference models to be tested, to look for the reference model to be tested that, among all the reference models to be tested, provides following one of said first optimization operations a view that corresponds best to the updated image.

A first optimization operation is preferably effected for each test of a reference model to be tested during the second optimization operation.

The first optimization operation and/or the second optimization operation, preferably the first optimization and the second optimization operation, preferably employ a metaheuristic, preferably evolutionist, method, preferably simulated annealing.

To modify initial reference model there is therefore tested a succession of reference models “to be tested”, the first reference model to be tested being the initial reference model.

The choice of a reference model to be tested preferably depends on the level of correspondence of the reference models “to be tested” previously tested using the updated image. That choice is preferably made by following a known optimization method, in particular chosen from metaheuristic optimization methods, preferably evolutionist, in particular simulated annealing methods.

All known optimization methods may be used. The updated image is preferably analyzed by means of a metaheuristic method, preferably evolutionist, preferably by simulated annealing, preferably by means of a method described in PCT/EP2015/074896.

“Metaheuristic” methods are known optimization methods. They are preferably chosen in the group formed by:
the evolutionist algorithms, preferably chosen from: evolution strategies, generic algorithms, differential evolution algorithms, distribution estimation algorithms, artificial immunity systems, shuffled complex evolution path recomposition, simulated annealing, ant colony algorithms, particle swarm optimization algorithms, taboo search, and the GRASP method,
the kangaroo algorithm,
the Fletcher and Powell method,
the noise method,
stochastic tunneling,
hill climbing with random restarts,
the cross entropy method, and
hybrids of the metaheuristic methods described above.

Analysis of the updated image may in particular employ metaheuristic methods as described in PCT/EP2015/074896. The method described in that document in particular enables it to determine the possible movements of the teeth represented in the updated image and/or modifications of their shape or of their appearance, and then to define attribute values accordingly, for example the value “abnormal” for the attribute “position of tooth No 13”.

The analysis may also use artificial intelligence algorithms, and in particular one or more neural networks using a learning base. These algorithms are well known.

A neural network may in particular be chosen from:
networks dedicated to image classification, for example convolutional neural networks (CNN),
   AlexNet (2012),
   ZF Net (2013),
   VGG Net (2014),
   GoogleNet (2015),
   Microsoft ResNet (2015),
   Caffe: BAIR Reference CaffeNet, BAIR AlexNet,
   Torch:VGG_CNN_S,VGG_CNN_M,VGG_CNN_M_2048,VGG_CNN_M_1 024,VGG_CNN_M_128,VGG_CNN_F,VGG ILSVRC-2014 16-layer,VGG ILSVRC-2014 19-layer,Network-in-Network (Imagenet & CIFAR-10),
   Google: Inception (V3, V4).
Networks dedicated to localization and detection of objects in an image, for example Object Detection Networks:
   R-CNN (2013),
   SSD (Single Shot MultiBox Detector: Object Detection Network), Faster R-CNN (Faster Region-based Convolutional Network method: Object Detection Network), p2 Faster R-CNN (2015), p2 SSD (2015).

The above list is not limiting on the invention.

The neural network is preferably trained by means of a learning base typically containing more than 10 000, preferably more than 50 000, preferably more than 100 000 records, each record containing a historical image, preferably a photo, of a “historical” dental arch and a “historical” description providing values for attributes of the historical image and in particular for attributes enabling evaluation of the dental situation of the patient.

In one embodiment the analysis uses only information provided by the updated image.

In a preferred embodiment the analysis effected in step c) uses not only the information contained in the updated image but also data relating to the target patient extracted from a database “DB_patients” based on the identifier of the target patient determined from the request. The analysis thereof is advantageously more precise.

The database “DB_patients” relating to patients may in particular contain general information on each patient, for example their age, information relating to the treatment in progress or to treatments undertaken in the past, and information relating to earlier exchanges between the patient and the analysis computer.

Analysis of the updated image enables determination of a value for at least one attribute relating to the dental situation of the target patient, or “dental attribute”.

The dental attribute may relate to the general dental situation, and for example take the value “satisfactory dental situation” or “unsatisfactory dental situation”, relating to a state of an orthodontic device represented in the updated image, and for example take the value “device intact” or “device damaged” relating to the state of a tooth, and for example take the value “abraded tooth”, “broken tooth” or “decayed tooth”.

The values of a dental attribute may also be quantitative. For example a value of an attribute may define how much a tooth has moved, has been abraded or has changed appearance.

The values of the dental attributes are then transmitted by the analysis computer to a “composition computer” that may be identical to or different from the analysis computer. All known communication means may be used for this purpose.

The composition computer may also be integrated into the mobile telephone.

In step d) the composition computer selects one or more relevant standard responses in the light of the dental situation. To this end it looks in a standard response base “DB_standard responses” for one or more standard responses addressing the dental attribute values determined in step c) and the composition rules.

The composition rules preferably enable selection as a function of data relating to the target patient and in particular relating to the treatment of the target patient.

For example, if a dental attribute value indicates detachment of an orthodontic aligner, such detachment may be interpreted in a different manner depending on the efficacy of the treatment in progress. For example, if the data relating to the target patient indicates that the detachment has occurred very rapidly, the computer is also able to select the relevant standard response: “please make an urgent appointment”. If on the other hand this detachment follows a very slow evolution in the position of the teeth the computer may select the relevant standard response: “please make an appointment with the orthodontist in the next two months”.

A plurality of relevant standard responses may be selected for the same aspect of the dental situation. For example, a first relevant standard response may relate to the detachment of the aligner and a second relevant standard response may relate to abnormal deformation of that aligner.

Different relevant standard responses may be selected for different aspects of the dental situation. For example, a relevant standard response may relate to detachment of the orthodontic aligner and a second relevant standard response may relate to the appearance of caries.

In step e) a response is composed by the composition computer, in accordance with the composition protocol and from the relevant standard response or responses determined in step d).

The composition step may be limited to the preparation of a message, for example an SMS message or an email repeating word for word the relevant standard response or responses.

Composition of the composed response may also include layout or formatting operations, for example to introduce a courtesy formula or the signature of the dental care professional who is monitoring the target patient.

Composition of the composed response may also include an operation of arranging the various relevant standard responses received and the introduction of linking phrases between those different relevant standard responses.

The data relating to the target patient may also be used to modify the standard response or responses in order for them better to match the target patient, for example translated into a language understood by the target patient.

Step e) preferably supplies a response close to that that a dental care professional would have been able to draft, preferably in a language register appropriate to the target patient.

In step f) the response composed by the composition computer is preferably submitted to a controller in order for it to validate it or to modify it, for example by adding to it. The controller is a dental care professional who may be the same as or different from the one who is treating the target patient. In particular, in a preferred embodiment, the controller monitors and/or modifies responses composed for patients of more than 1, more than 5, more than 10 or even more than 50 dental care professionals. In one embodiment the controller does not have a specific patient base.

In one embodiment the relevant standard response is or is not verified by a controller, depending on the relevant standard response.

In step g), if the composition computer is not integrated into the mobile telephone, the composed response is sent to the mobile telephone by the composition computer, preferably by means of a mobile telephone network.

The composed response is then presented on the mobile telephone. The target patient advantageously therefore receives a rapid response to their request, well matched to their dental situation.

A composed response is preferably presented systematically to the patient for each occurrence of step a).

An information message is more preferably also presented to the dental care professional who is monitoring the patient. The information message may be a copy of the composed response or be personalized, in accordance with the second principal aspect of the invention. A method in accordance with the invention therefore advantageously enables the professional systematically to obtain a report on the dental situation of the patient and to act accordingly.

The method may also serve to inform the patient in order for them to improve their oral and dental hygiene, for example to teach them how to brush their teeth better.

As is now clearly apparent, the composed response therefore depends on:
the analysis of the updated image;
the list of standard responses available in the standard responses base;
the composition protocol.

A method in accordance with the invention therefore makes it possible to respond to an inquiry relating to the dental situation of a target patient rapidly, precisely and without stress for the dental care professional.

The method may in particular be used to control the progress of an orthodontic treatment, after fitting a device intended to correct the positions of the teeth of the target patient, a so-called “active orthodontic device”.

The method may also be employed after an orthodontic treatment to verify that the positions of the teeth have not evolved in an unfavorable manner (“recurrence”), in particular after fitting a device intended to hold the teeth in position, a so-called “passive orthodontic device”.

### EXAMPLE

In a preferred embodiment a pre-parametered protocol model is presented to the dental care professional when they wish to apply a composition protocol to compose and send a composed request to a specific target patient.

The protocol model preferably lists standard responses classified for example as a function of the framework within which the target patient is being monitored, for example as a function of the possible orthodontic device worn by the patient (orthodontic aligner, device with arc wires and attachments, retainer device, etc.) or outside any orthodontic treatment (general intra-oral evaluation).

The standard responses are further preferably classified within the above categories, into sub-categories, for example as a function of the element of the orthodontic device concerned. For example, in the category relating to orthodontic devices with arc wires and attachments the standard responses may be classified into sub-categories “arc wire” and “attachments” according to whether they relate to the arc wire or to the attachments, respectively.

The standard responses are further preferably classified, within the above sub-categories, into rubics, for example as a function of the nature of the anomaly. For example, in the sub-category relating to the attachments, the standard responses may be classified into rubrics “attachment detached” and “attachment damaged” according to whether they relate to detachment of or damage to the attachment, respectively.

For each standard response the dental care professional is preferably able:
to choose the alert level, for example to choose to do nothing, to inform the target patient, or to alert the patient with the addition of a high importance indicator; and/or
to choose the teeth to be excluded from the analysis in step c) and therefore for which the composed response will not include any information; and/or
to choose a message for the target patient, preferably in a predefined list; and/or
to choose a message for the dental care professional, preferably in a predefined list.

Independently of the standard responses, the preparametered protocol model preferably also enables the dental care professional:
to define the frequency at which the patient will be invited for scans in the absence of specific instructions addressed to the target patient by the dental care professional (step a));
to define the frequency at which the patient will be invited for scans in the absence of specific instructions addressed to the target patient by the dental care professional, for example to indicate to them that they must change orthodontic groove (step a)).

### DETAILED DESCRIPTION OF A SECOND EMBODIMENT

A method in accordance with the second principal aspect of the invention may have one or more of the optional features of the method in accordance with the first principal aspect of the invention described hereinabove unless they are incompatible.

A method in accordance with the second principal aspect of the invention is intended to present to the dental care professional a composed response thanks to a “professional” composition protocol identical to or preferably different from the one who is to compose the composed response intended for the target patient.

The professional composition protocol preferably enables definition of thresholds from which a composed response must be composed and transmitted to the dental care professional. For example, the professional composition protocol makes it possible to determine if a dental situation, for example a non-pathological malocclusion, must be reported to the dental care professional or not.

The professional composition protocol preferably enables definition of a level of detail for the composed response, for example to specify values of movement or of deformation of the teeth.

The professional composition protocol preferably makes it possible to determine if the composed response must include medical recommendations, for example to advise a particular approach such as the production of an image by tomography, or the modification of an orthodontic device, or changing the orthodontic device of the target patient, or more generally modification of the treatment.

In one embodiment the composed response intended for the dental care professional does not provide a diagnosis and/or does not include any medical recommendation.

The professional composition protocol preferably enables determination if the composed response has to include recommendations for modifying the scheduling of appointments with the target patient and/or modifying the composition protocol used to compose the composed responses addressed to the target patient and/or to modify the frequency of reminders addressed to the target patient for them to carry out a step a).

The professional composition protocol preferably also enables determination of the format of the composed response, as described above.

The professional composition protocol preferably also enables definition of a list of destinations of the composed response, or even definition of specific documents to send to the addressees.

The professional composition protocol preferably also makes it possible to inform the dental care professional that they have to undertake new tasks. For example, it may lead to composing a composed response making it possible to inform a dental nurse that they must prepare the next care session in accordance with the dental attribute values determined in step c). The nurse may for example be informed that they must prepare a tray for the next care session, in particular to dispose thereon the disposable care equipment necessary for that session.

The professional composition protocol preferably also enables automatic undertaking of new tasks. For example, the professional composition protocol may lead to the generation of an email or of a letter to a prosthetist so that they fabricate a prosthesis or to the target patient in order for them to make an appointment. The professional composition protocol may also, for example, lead to the generation of an order for orthodontic device parts, for example attachments, and/or an orthodontic device, for example a groove.

The method may be used to monitor the target patient, in particular outside orthodontic or dental care treatment periods. Dental problems, for example the onset of caries or necrosis of a soft tissue or infection, may therefore be detected early, or even anticipated, in particular if the analysis in step c) uses artificial intelligence.

Using a professional composition protocol enables the dental care professional to save time, and also to obtain a medical summary well matched to their preferences.

### DETAILED DESCRIPTION OF A THIRD EMBODIMENT

The method in accordance with the third principal aspect of the invention may have one or more of the optional features of the method in accordance with the first and second principal aspects of the invention described hereinabove unless they are incompatible.

A method in accordance with the third principal aspect of the invention is intended to present to a third party, in particular an insurance professional, a response composed thanks to a composition protocol. That composition protocol is preferably determined by the third party. If a response is also composed for the target patient or the dental care professional the “third party” composition protocol, that is to say the one used to prepare the composed response intended for the third party, may be identical to or preferably different from that used to compose the composed responses intended for the target patient and for the dental care professional.

A method in accordance with the invention is particularly useful for a target patient to be able, without stress, and in particular without having to attend a dental care professional's surgery, to send the insurance professional information relating to their dental situation.

Depending on the situation, the dental care professional is then able:
to prepare an insurance policy;
to determine a treatment for the teeth of the target patient;
to verify that a treatment of the teeth of the target patient is being monitored and/or is effective and/or is covered by an insurance policy taken out by the target patient;
as a function of a treatment of the teeth of the target patient, to determine and to recommend to the target patient a dental care professional;
to verify that the declarations of the target patient conform to their dental situation.

In one embodiment the composition protocol is determined in such a manner as to compose both a composed response for the target patient and/or a composed response addressed to the insurance professional and/or a composed response addressed to the dental care professional.

In one embodiment the composition protocol leads to the insertion of advertising into the composed response, in particular into the composed response intended for the target patient. The composition protocol preferably determines the advertising as a function of data relating to the target patient that cannot be determined by the analysis of the dental situation in step c).

In one embodiment the composition protocol leads to the insertion into the composed response intended for the target patient of a recommendation of a dental care professional.

Of course, the invention is not limited to the embodiments described hereinabove and represented.

Generally speaking, a composed response and/or an information message may be presented to the target patient and/or to any third party, for example an insurance professional and/or a dental care professional, for example via a web interface.

Moreover, the target patient is not limited to a human being. A method in accordance with the invention may be used for another animal.

A method according to the invention is not necessarily used for diagnostic purposes. It may for example be used for teaching or information or commercial purposes.

1. A method for preparing a composed response from a value for at least one dental attribute relating to the dental situation of a target patient, said method including the following steps:
d) selection by a composition computer of at least one relevant standard response from a base of standard responses and as a function of said value of said at least one dental attribute;
e) composition of a composed response by the composition computer on the basis of said at least one relevant standard response;
f) optionally, validation and/or modification of the composed response by a controller;
g) presentation of the optionally validated and/or modified composed response, the selection in step d) and the composition in step e) being determined according to composition rules of a composition protocol,
in which method, before step e), the composition protocol is selected in a protocol models base and/or is created by selecting a protocol model in a protocol models base and then modifying same.

2. The method as claimed in claim 1, in which the composition rule is configured to operate on said composition as a function of data relating to the target patient that cannot be determined by the analysis of the dental situation of the target patient.

3. The method as claimed in claim 1, including a step f), in which in the step f) the controller is a dental care professional different from a dental care professional who is monitoring the target patient, and in which the controller checks and/or modifies composed responses for patients of more than 5 dental care professionals.

4. The method as claimed in claim 1, in which before step e),
a dental care professional monitoring the target patient, or
an insurance professional, or
an organization having a centralized computer program to execute at least steps d) and e) on the basis of a plurality of requests each containing a value of said at least one dental attribute,
selects the composition protocol in said protocol models base and/or creates the composition protocol by selecting a protocol model in a protocol models base, and then modifying same.

5. The method as claimed in claim 1, in which at least one composition rule is chosen in the group formed by:
a filter determining if a standard response may or may not be integrated into the composed response;
a composition rule imposing a format on the relevant standard response;
a composition rule determining the position of each relevant standard response as a function of its content or of a category to which it belongs;
a conditional composition rule.

6. The method as claimed in claim 5, in which the conditional composition rule:
has an effect that depends on the dental situation of the target patient, and/or
has an effect that depends on the standard response to which it applies, and/or
modifies the pertinent standard response or the composed response as a whole, and/or
takes account of past events, and/or
is applied to the composed response and depends on data relating to the target patient, and/or
has an effect that depends on earlier exchanges with the target patient, and/or
has an effect that depends on an arbitrary choice by a dental care professional who is monitoring the target patient or an insurance professional.

7. The method as claimed in claim 1, in which at least one composition rule is configured to operate on said composition as a function of said at least one relevant standard response selected in step d) and/or as a function of data relating to the target patient and/or of data relating to a dental care professional who is monitoring the patient and/or of data relating to an insurance professional.

8. The method as claimed in claim 1, in which, in accordance with a composition protocol identical to or different from that used in step d) and step e), the composition computer composes a medical summary as a function of said value of said at least one dental attribute and presents said medical summary to a dental care professional monitoring the target patient.

9. A method for evaluating a dental situation of a target patient, optionally monitored by a dental care professional, said method including the following steps:
a) acquisition by means of a mobile telephone of at least one updated image representing a dental arch of the target patient;
b) preparation by the mobile telephone of a request including the updated image and transmission of the request to an analysis computer;
c) analysis of the request by the analysis computer in such a manner as to determine a value for at least one dental attribute relating to the dental situation of the target patient and transmission of the value of the dental attribute to a composition computer;
d)-g) preparation of a composed response in accordance with a method as claimed in claim 1.

10. The method as claimed in claim 9, in which, in step g), the composition computer transmits the composed response, optionally validated and/or modified,
to the mobile telephone, and the mobile telephone presents the optionally validated and/or modified composed response, and/or
to a dental care professional who is monitoring the patient, and/or
to an insurance professional.

11. The method as claimed in claim 9, in which the target patient is monitored by a dental care professional and the composition protocol is common to all the patients of a group of patients of the dental care professional.

12. The method as claimed in claim 9, in which the analysis computer is programmed, in step c)
to measure the value of the dental attribute in the updated image; and/or
to segment the updated image and to measure the value of the dental attribute over a segment of the updated image; and/or
on the basis of the updated image, to look for additional information specific to the patient and to determine the value of the dental attribute from said additional information.

13. The method as claimed in claim 12, in which the analysis computer is programmed, in step c), to determine a three-dimensional digital model of the dental arch, called an “updated reference model”, observable in a view that can be superposed on and in register with the updated image, and then to determine the value of the dental attribute from measurements on the updated reference model.

14. The method as claimed in claim 13, in which the analysis computer is programmed to determine the updated reference model by deformation of an initial reference model.

15. The method as claimed in claim 9, in which, in step c), the value of the dental attribute qualifies or quantifies the general dental situation of the target patient and/or an orthodontic situation.

16. The method as claimed in claim 15, in which, in step c), the value of the dental attribute qualifies or quantifies a state of the orthodontic device represented on the updated image and/or a state of a tooth of the target patient represented on the updated image and/or an oral-dental hygiene of the target patient.

17. The method as claimed in claim 9, in which, in step c), said analysis is effected by means of one or more neural networks.

18. The method as claimed in claim 9, in which, before step d), the composition computer evaluates the reaction of the target patient following the sending to the target patient of an earlier composed response generated in an earlier step g) and then, in step d), selects said at least one standard response as a function of the evaluation of said reaction.

19. The method as claimed in claim 9, in which the target patient is monitored by a dental care professional and in which, before step e), the dental care professional configures the composition protocol in such a manner as to select a region of the mouth of the target patient so that the analysis in step c) and/or the selection of the relevant standard response in step d) relate(s) only to said region.

20. The method as claimed in claim 9, in which, in step d), the composition computer selects at least one standard response as a function of data relating to the target patient that cannot be determined by the analysis of the dental situation in step c).

21. The method as claimed in claim 9, in which the analysis computer and the composition computer are integrated into the mobile telephone or in which the analysis computer and/or the composition computer are remote from the mobile telephone.

22. The method as claimed in claim 9, in which the composed response is transmitted to a third party and then the third party:
prepares an insurance policy; and/or
determines a treatment of the teeth of the target patient; and/or
verifies that the treatment of the teeth of the target patient is being monitored and/or is effective and/or is covered by an insurance policy taken out by the target patient; and/or
as a function of a treatment of the teeth of the target patient, determines and recommends to the target patient a dental care professional; and/or
verifies that the declarations of the target patient conform to their dental situation.
